(19) United States
(12) Patent Application Publication
(10) Pub. No.: US 2026/0240569 A1
(43) Pub. Date: Aug. 20, 2026

(54) TEMPORAL BONE IMPLANT AND METHOD FOR CONFIGURING AND/OR MANUFACTURING A TEMPORAL BONE IMPLANT

(71) Applicant: Karl Leibinger Asset Management GmbH & Co. KG, Mühlheim an der Donau (DE)

(72) Inventors: Adem AKSU, Schwenningen (DE); Frank REINAUER, Emmingen-Liptingen (DE)

(21) Appl. No.: 19/539,595

(22) Filed: Feb. 13, 2026

(30) Foreign Application Priority Data
Feb. 14, 2025 (DE) 102025105585.8

(51) Int. Cl. A61B 17/70; A61B 17/16; A61B 17/80; A61F 2/30; A61F 2/46

(52) U.S. Cl. CPC A61B 17/701; A61B 17/1675; A61B 17/7082; A61B 17/8004; A61B 17/8061; A61F 2/4603; A61F 2002/3093

(57) ABSTRACT

A temporal bone implant (SI) for at least partially reconstructing a posterior wall of an external auditory canal includes a first portion (A1) that is shaped as a tube or a tube segment and is configured to form at least one wall portion of the posterior wall by covering a void in a temporal bone and thereby separate the auditory canal from the void. The first portion (A1) transitions into a second portion (A2) that is oriented transversely to the first portion (A1) and is plate-shaped. This second portion (A2) is configured to be attached to a mastoid of the temporal bone and to cover the void together with the first portion (A1).

### CROSS-REFERENCE TO RELATED APPLICATION

The present application is related and has right of priority to German Patent Application No. DE 102025105585.8 filed on Feb. 14, 2025, which is incorporated by reference in its entirety for all purposes.

### TECHNICAL FIELD

The invention relates generally to a temporal bone implant, which is provided for at least partially restoring a posterior wall of an external auditory canaThe invention further relates generally to a method for configuring and/or manufacturing a temporal bone implant.

### BACKGROUND

In chronic diseases in the region of the middle ear, surgical treatment in the form of a radical mastoidectomy may be necessary in certain cases. In a radical mastoidectomy on a temporal bone (os temporale), a mastoid (pars mastoidea) is cleaned out, with usually partial removal of the posterior auditory canal wall in order to form an open cavity from an attic of the middle ear, the mastoid, and the auditory canal. This cavity is frequently also referred to as the mastoid cavity or the radical cavity. A radical mastoidectomy can be necessary in the case of a pronounced cholesteatoma, which is chronic suppurative otitis media of the middle ear. The aim of the radical mastoidectomy is total removal of a matrix of the cholesteatoma. In addition to a reconstruction of the posterior auditory canal wall by cartilage (skin) pieces (sometimes referred to as the canal wall-up technique), implants are also known, which can be used to restore the posterior wall of the auditory canal.

DE 31 17 025 A1 discloses an implant, which is provided for the partial or total reconstruction of a posterior auditory canal wall which is to be partially or totally removed and replaced by an implant due to pathological conditions. The implant has a body shaped as a tube or a tube segment, which is to reproduce the anatomical conditions of the posterior auditory canal wall due to its shape.

### BRIEF SUMMARY

Example aspects of the present invention provide a temporal bone implant, by which restoration of a posterior wall of an auditory canal requiring minimal aftercare is to be possible.

In example aspects, a temporal bone implant, which is provided for an at least partial restoration of a posterior wall of an external auditory canal, has a first portion. This first portion is shaped as a tube or a tube segment and is designed to form at least one wall portion of the posterior wall by covering a void in a temporal bone and thereby separate the auditory canal from the void.

The implant according to example aspects of the invention is therefore designed for use in a temporal bone (os temporale) and is used in this application to at least partially restore a posterior wall of the external auditory canal. Preferably, the temporal bone implant is used in the case of a surgically formed mastoid cavity, which is frequently also referred to as a radical cavity and has been formed by a radical mastoidectomy.

According to example aspects of the invention, the temporal bone implant is provided with a first portion, which is designed to cover a void in the temporal bone and, due to this coverage, separate the external auditory canal, which is to be restored, from the void. Due to the separation, the first portion forms a part of the posterior osseous auditory canal wall of the external auditory canal (meatus acusticus externus osseus). Therefore, when the temporal bone implant is used, the first portion delineates the external auditory canal from the void formed in the temporal bone. In particular, when the temporal bone implant is used, the first portion thereof covers a void in a tympanic part (pars tympanica) of the temporal bone and the first portion separates this void in the tympanic part from the auditory canal.

The first portion of the temporal bone implant is shaped as a tube or a tube segment, which is to be understood within the meaning of the invention to mean that a shape of the first portion is designed similarly to a tube or similarly to a segment formed by subdividing a tube along a longitudinal direction of the tube. This shape of the first portion is selected in order to achieve the delineation of the external auditory canal from the void in the temporal bone and, in addition, to form at least one wall portion of the posterior wall of the external auditory canal. Specifically, the shape of the first portion as a tube or a tube segment can be configured with different internal diameters and, depending on the course of the external auditory canal to be reconstructed, can also be shaped as a funnel or a funnel segment.

Preferably, the temporal bone implant has an inner face with which the temporal bone implant is to be placed on the temporal bone and which can thus also be referred to as the bone face. Situated opposite the inner face is an outer face of the temporal bone implant, on which outer face the auditory canal lies, which auditory canal is to be separated via the first portion. The outer face in the region of the first portion can therefore also be referred to as the auditory canal face.

Preferably, the first portion forms, in its region, a closed partition of the temporal bone implant, i.e. on the first portion, there are no passages formed between the outer face and the inner face of the temporal bone implant. As a result, when the auditory canal is separated from the void via the first portion, bodily fluids from the void can be prevented from reaching the external auditory canal through the first portion. This is the case because this would increase the risk of recurrent inflammation.

In example aspects of the invention, the first portion transitions into a second portion, which is oriented transversely to the first portion and is plate-shaped. The second portion is provided to be attached to a mastoid of the temporal bone and to cover the void together with the first portion. In other words, the first portion is therefore abutted by a second portion of the temporal bone implant, wherein this second portion is oriented extending transversely to the first portion and is designed similarly to a plate. In the temporal bone implant, the second portion is designed to be attached to a mastoid (pars mastoidea) of the temporal bone and to cover the void in the temporal bone together with the first portion.

The embodiment according to example aspects of the invention has the advantage that reliable coverage of a void in a temporal bone can be achieved by the first portion shaped as a tube or a tube segment and by the plate-shaped second portion, which void is of the type that arises in particular when a radical mastoidectomy is performed. This is the case because, whereas the first portion can cover in particular a void in the region of a tympanic part of the temporal bone, the second portion can preferably cover a void in the region of the mastoid of the temporal bone. In addition, on the second portion, the temporal bone implant can be reliably attached to the temporal bone, namely to the remaining portion of the mastoid of the temporal bone. Furthermore, via the first portion, the auditory canal can be separated from the void and, thereby, the posterior osseus wall of the external auditory canal can be reconstructed. It is particularly advantageous when the first portion forms a closed partition, since this at least impedes, preferably entirely prevents, passage of bodily fluids from the void into the auditory canal. Overall, a temporal bone implant can therefore be obtained, which is suitable for sealing a surgically formed mastoid cavity or radical cavity and requires minimal aftercare due to the separation of the auditory canal from the void. In particular, the risk of recurrent inflammation after treatment of a cholesteatoma can be reduced as a result.

The second portion is plate-shaped, which is preferably to be understood within the meaning of the invention to mean that the second portion has a substantially flat shape similar to a plate, in that the second portion extends in a coordinate system primarily along two coordinate axes but can have one or more curvatures in order to follow the anatomy. The second portion is provided to be attached with the inner face of the temporal bone implant to the mastoid of the temporal bone.

The second portion of the temporal bone implant is provided not only to be attached to the mastoid but also to cover the void in the temporal bone together with the first portion when the temporal bone implant is used. Whereas the first portion is used in particular to cover the void in the region of the tympanic part of the temporal bone, the second portion is preferably provided for covering the void in the region of the mastoid of the temporal bone. Most preferably, the first portion and the second portion of the temporal bone implant are designed to completely cover the void in the temporal bone together.

Preferably, the first portion and the second portion of the temporal bone implant are formed in one piece, whereby the temporal bone implant is formed as a single piece. Most preferably, the first portion and the second portion have been formed in an additive manufacturing process, in particular within the framework of a 3D printing process. Alternatively, preferably however, additionally, the first portion and the second portion are entirely or partially formed of a metal, such as titanium, stainless steel, magnesium, or molybdenum, or of a metal alloy, for example a titanium alloy, or of a plastic, in particular a polymer, for example of polyether ether ketone (PEEK), polyethylene (PE), polycaprolactone (PCL), or of a ceramic, of beta-tricalcium phosphate (β-TCP), of zirconia, of hydroxyapatite (HA), or of combinations of the aforementioned materials. In addition, non-porous surfaces of the two portions or porous surfaces of the two portions can be formed or sub-portions of the two portions can be porous and sub-portions can be non-porous.

According to one example embodiment of the invention, the second portion has an attachment part and a cover part. The attachment part forms, on an inner face with which the temporal bone implant is to be placed on the temporal bone, a contact face with which contact is to be made with the mastoid for attachment to the mastoid. The cover part is provided for covering the void. Thus, the second portion can be reliably configured to be attached to the temporal bone and to cover the void. Preferably, the attachment part peripherally partially surrounds the cover part, such that, at the attachment part the temporal bone implant can be attached to the mastoid in a manner surrounding the void.

In a development of the aforementioned example embodiment, a plurality of receiving openings can be introduced into the attachment part of the second portion, which receiving openings extend from the inner face to an outer face of the temporal bone implant facing away from the inner face and are used to guide attachment mechanisms through. Advantageously, the temporal bone implant can be attached to the temporal bone at these receiving openings. Preferably, the receiving openings are configured as circular openings, which can be introduced into the attachment part of the second portion in an abrasive or metal-cutting manner as bores. If the second portion was produced within the framework of an additive manufacturing process, the receiving openings may have also been defined within the framework of the additive manufacturing. The attachment mechanisms that are used can be bone screws for screwing the temporal bone implant to the temporal bone or also a suture for suturing to the temporal bone.

Alternatively or additionally, passages are introduced into the cover part, which extend from the inner face to an outer face of the temporal bone implant facing away from the inner face. These passages can be used as suture holes for attaching skin to the cover part of the second portion, in particular for the case in which the second part if formed of a material on which growth of tissue is made difficult or impossible.

According to one possible example embodiment of the invention, the first portion forms, on an inner face with which the temporal bone implant is to be placed on the temporal bone, a sealing face which is provided to be placed on the temporal bone. Advantageously, the auditory canal can thus be sealed with respect to the void in the temporal bone. It is particularly advantageous when, in addition, a coating is applied on the sealing face, since this enables a particularly reliable seal to be realized. This coating can be a flexible material such as porous polyether ether ketone (PEEK), porous polyethylene (PE), polycaprolactone (PCL), or the like. In addition, the coating could be optionally designed to be absorbable.

In a development of the aforementioned possible example embodiment, the sealing face can be at least partially formed on a periphery of the first portion and provided for sealing the auditory canal with respect to the void via contact with the temporal bone. More preferably, a continuous attachment to the temporal bone is achieved at the sealing face in the region of the first portion in order to obtain a seal that is as closed as possible when the auditory canal is separated from the void.

According to an additional possible example embodiment of the invention, the first portion and the second portion are shaped in a patient-specific manner. This has the advantage that the first portion and the second portion, and thus also the temporal bone implant overall, are adapted to the temporal bone of the patient. Preferably, the first portion and the second portion are modeled after bone segments which are to be removed from the temporal bone of the patient. In particular, contours of the temporal bone have been determined for this purpose in advance within the framework of a medical imaging process, such as CT.

Example aspects of the invention also relate to a method for configuring and/or manufacturing a temporal bone implant, which is provided for an at least partial restoration of a posterior wall of an external auditory canal. In the temporal bone implant, a first portion shaped as a tube or a tube segment is designed to form at least one part of the posterior wall by covering a void in a temporal bone and thereby separate the auditory canal from the void. In the temporal bone implant, in addition to the first portion, a second portion is configured, which is plate-shaped and oriented transversely to the first portion, wherein the second portion is designed to be attached to a mastoid of the temporal bone and to cover the void together with the first portion.

Within the context of the method according to example aspects of the invention, a configuration or a manufacture, or both a configuration and a manufacture, of a temporal bone implant is/are therefore carried out. A “configuration” is to be understood to mean in particular planning and designing the temporal bone implant, i.e. creating a design or a template for manufacturing the temporal bone implant. “Manufacture” is intended to mean production of the temporal bone implant, i.e. the physical embodiment of the temporal bone implant. In particular, both the configuration and the manufacture of a temporal bone implant are carried out within the framework of the method according to example aspects of the invention. Most preferably, the manufacture is carried out in an additive manufacturing process, in particular by a 3D printing process.

Advantageously, a temporal bone implant can therefore be configured and/or manufactured, by which reliable coverage of a void is achievable, which void is of the type that arises in particular when a radical mastoidectomy is performed. Furthermore, the configured and manufactured temporal bone implant can be used to achieve a separation of the auditory canal from the void via the first portion and thus reconstruct a posterior osseus wall of the auditory canal (meatus acusticus externus osseus). Overall, a temporal bone implant can thus be obtained, which is suitable for sealing a surgically formed mastoid cavity or radical cavity and requires minimal aftercare due to the separation of the auditory canal from the void.

Preferably, the temporal bone implant is also formed with respect to configuration and/or with respect to creation according to at least one of the above-described variants of a temporal bone implant according to example aspects of the invention.

In a development of the method according to example aspects of the invention, the first portion and the second portion can be modeled during the configuration thereof after bone segments which are to be removed from the temporal bone of the patient. As a result, a patient-specific adaptation of the temporal bone implant can be achieved. Alternatively, preferably however, additionally, the first portion and the second portion are configured according to previously determined contours of the temporal bone, wherein this determination has been carried out in particular within the framework of a medical imaging process, such as a CT.

According to an additional possible embodiment of the method according to example aspects of the invention, a surgical template is also planned and/or manufactured together with the temporal bone implant. Advantageously, this surgical template can then be used to perform the radical mastoidectomy, wherein precise removal of segments of the temporal bone is thereby achievable.

### BRIEF DESCRIPTION OF THE DRAWINGS

An advantageous example embodiment of the invention, which is explained in the following, is represented in the drawings, wherein:

FIG. 1 shows a perspective representation of a temporal bone implant according to a preferred example embodiment;

FIG. 2 shows a perspective view of a part of a cranium in the region of a temporal bone with a marked region of the temporal bone to be removed;

FIG. 3 shows an additional perspective representation of the region of the cranium from FIG. 2, with an applied surgical template;

FIG. 4 shows an additional perspective representation of the region of the cranium from FIG. 2 after removal of the region of the temporal bone; and

FIG. 5 to 7 show different views of the cranium after insertion of the temporal bone implant.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

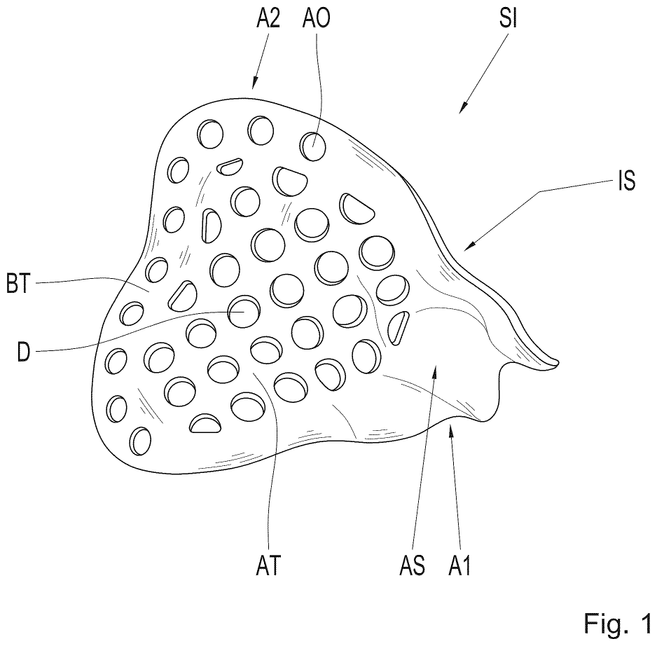

FIG. 1 shows a perspective representation of a temporal bone implant SI, which is configured according to a preferred example embodiment of the invention. This temporal bone implant SI has been manufactured in one piece within the framework of an additive manufacturing process, wherein this manufacture has been carried out by a 3D printing process.

In the present example case, the temporal bone implant SI is formed of titanium, wherein, alternatively, one other metal material, such as stainless steel or magnesium, a metal alloy, for example a titanium alloy, a plastic, in particular a polymer, for example polyether ether ketone (PEEK), polyethylene (PE), polycaprolactone (PCL), a ceramic, beta-tricalcium phosphate (β-TCP), zirconia, or hydroxyapatite (HA), and a combination of these materials may have also been used, however.

The temporal bone implant SI includes a first portion A1 and a second portion A2. The first portion A1 is shaped as a tube segment by designing the first portion A1 with a curvature directed toward an outer face AS of the temporal bone implant SI. The first portion A1 transitions into the second portion A2, which is oriented transversely to the first portion A1 and is planar.

The second portion A2 has a cover part AT and an attachment part BT, which surrounds the cover part AT on an outer periphery. In the attachment part BT, a plurality of receiving openings AO is designed as circular openings, each of which extends from the outer face AS to an inner face IS opposite the outer face AS of the temporal bone implant SI and passes completely through the attachment part BT.

As is apparent in FIG. 1, the cover part AT of the second portion A2 is provided with passages D, which pass completely through the cover part AT of the second portion A2 from the outer face AS to the inner face IS. The first portion A1 of the temporal bone implant SI, however, is designed as a solid wall and has no passages or openings between the outer face AS and the inner face IS.

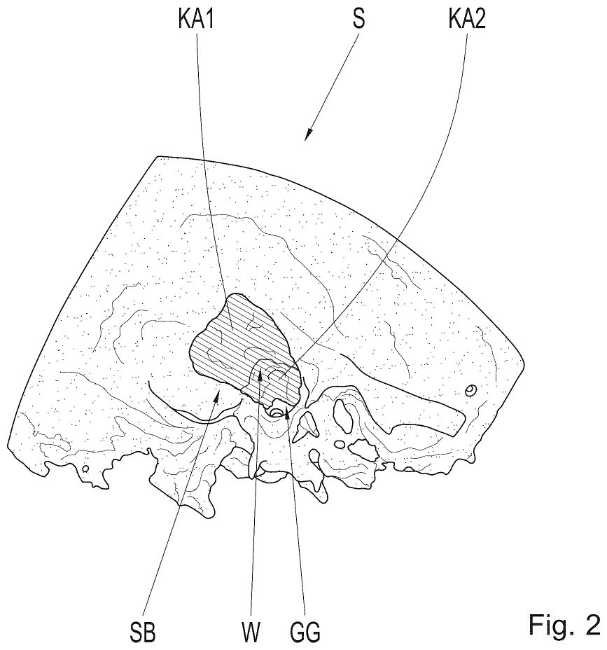

The temporal bone implant SI represented in FIG. 1 has been configured and manufactured for the actual, patient-specific application in the region of a temporal bone SB of a cranium S. To this end, the shape of this cranium S was determined in advance by a medical imaging process, in particular by CT, taking into account a 3D model of the cranium S partially represented in each of FIGS. 2 to 7. The temporal bone implant SI is formed in a patient-specific manner by configuring the two portions A1 and A2 of the temporal bone implant SI in accordance with bone portions KA1 and KA2, which have been declared regions of the temporal bone SB of the cranium S to be removed within the framework of a radical mastoidectomy and are indicated with shading in FIG. 2. The radical mastoidectomy is to be carried out in particular in order to be able to totally remove a cholesteatoma in the region of an external auditory canal GG.

In the present example case, the bone portion KA1 to be removed is a portion of a mastoid (pars mastoidea) of the temporal bone SB, whereas the bone portion KA2 to be removed is a portion of a tympanic part (pars tympanica) of the temporal bone SB and forms a part of the posterior osseus wall W of the external auditory canal GG.

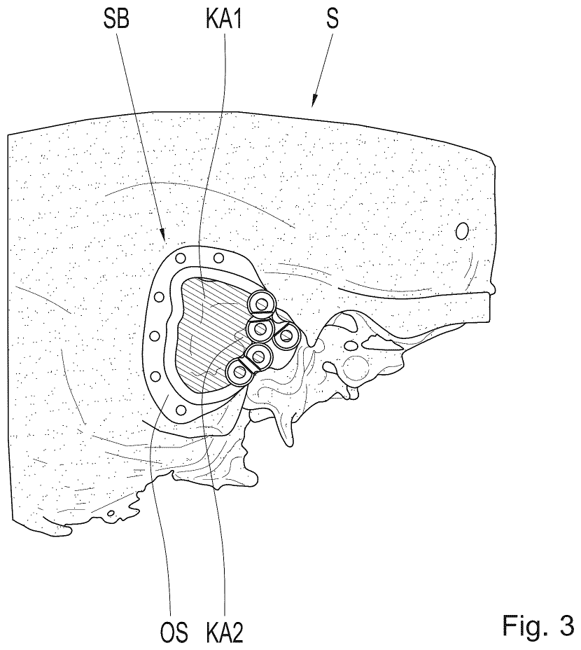

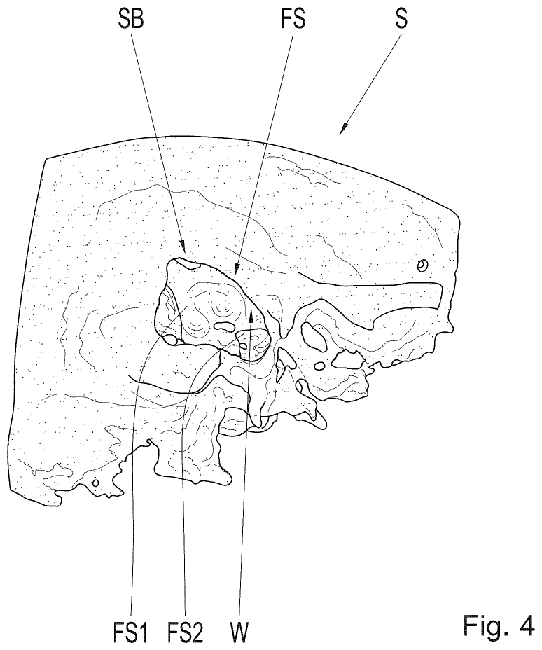

FIG. 3 shows the cranium S with an applied surgical template OS, which, together with the temporal bone implant SI, has been configured in a patient-specific manner on the basis of the determined shape of the cranium S. This surgical template OS, as is apparent with reference to FIG. 3, is designed to be attached to the temporal bone SB and, as a sawing and drilling template, defines the bone portions KA1 and KA2 to be removed. Furthermore, the cranium S is shown in FIG. 4 after removal of the bone portions KA1 and KA2, whereby a void FS has formed in the temporal bone SB. Due to the fact that the bone portions KA1 forms a portion of the mastoid and the bone portion KA2 forms a portion of the tympanic part, the void FS has a void part FS1 in the region of the mastoid and a void part FS2 in the region of the tympanic part, wherein, due to the latter, a portion of the wall W has been removed.

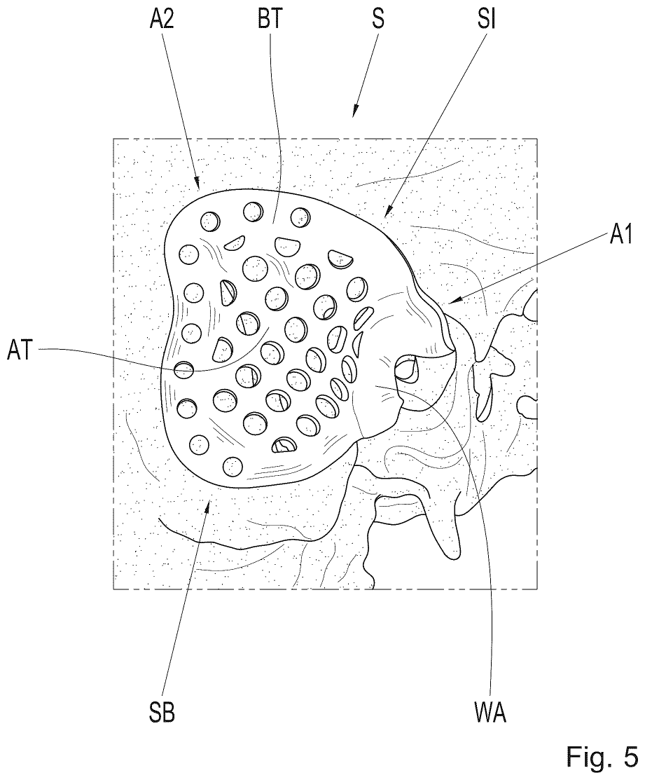

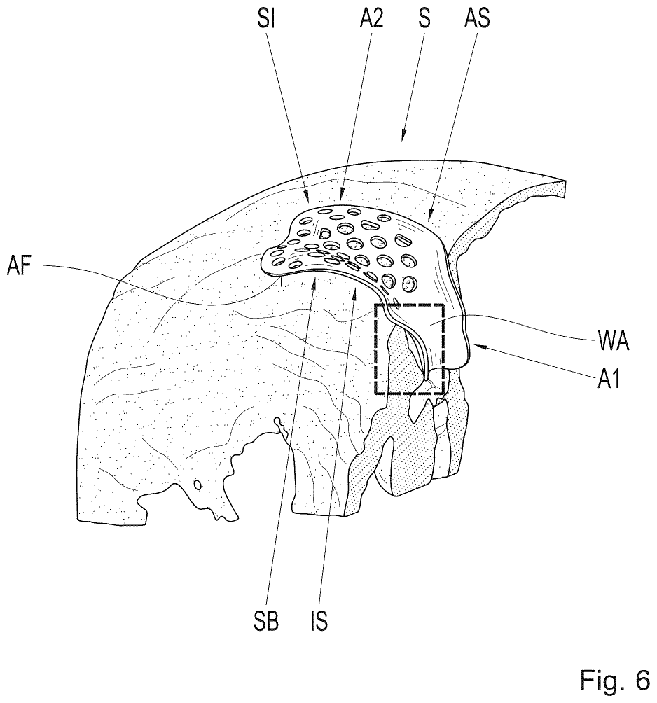

In FIG. 5 to FIG. 7, the cranium S is shown in each example case with the temporal bone implant SI arranged on the temporal bone SB, with the attachment part BT of the second portion A2 of the temporal bone implant having been placed on the mastoid of the temporal bone SB. As is apparent in particular with reference to FIG. 6, this placement has been carried out on the inner face IS of the temporal bone implant SI at a contact face AF with which the attachment part BT is equipped on the inner face IS. At the receiving openings AO of the attachment part BT, the temporal bone implant SI can be attached to the temporal bone SB, for the purpose of which corresponding attachment mechanisms (not represented in the figures) are to be guided through the receiving openings AO. Within the context of example aspects of the invention, these attachment mechanisms can be bone screws for screwing or a suture for suturing to the temporal bone SB. In addition, the passages D in the cover part AT can be used as suture holes for suturing the temporal bone implant SI to tissue, in particular skin.

Whereas the void part FS1 is covered via the cover part AT of the second portion A2, the first portion A1 covers the void part FS2 and thereby also forms a wall portion WA of the posterior wall W. As a result, the first portion A1 separates the external auditory canal GG from the void FS. Via the two portions A1 and A2, complete coverage of the void FS is also obtained.

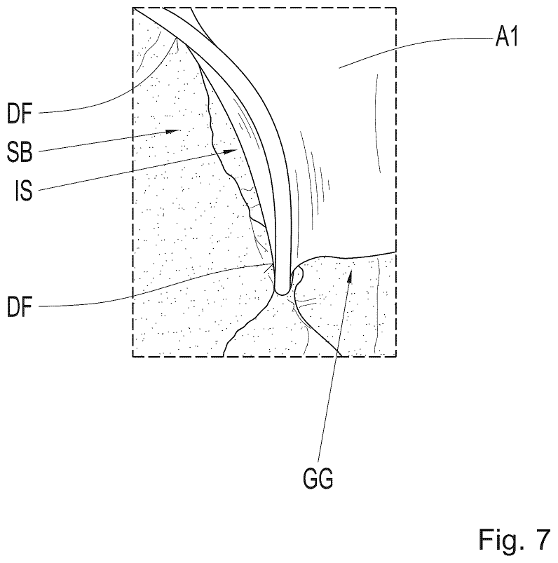

As is also apparent with reference to a detailed view in FIG. 7, the first portion A1 forms a sealing face DF on the inner face IS on a periphery, at which sealing face the first portion A1 lies peripherally against the temporal bone SB at an outer border of the void part FS2 and, as a result, seals the external auditory canal GG with respect to the void FS. Due to this peripheral contact and the example embodiment of the first portion A1 as a solid wall, by the temporal bone implant SI, bodily fluids are prevented from entering the external auditory canal GG via the void FS. In order to further improve the seal, the first portion A1 can also be provided with a coating on the sealing face DF.

By the example embodiment according to example aspects of the invention, a temporal bone implant can therefore be provided, by which restoration of a posterior wall of an external auditory canal requiring minimal aftercare is possible.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

### List of Reference Characters

SI temporal bone implant
A1 first portion
A2 second portion
AS outer face
AT cover part
BT attachment part
AO receiving openings
IS inner face
D passages
SB temporal bone
S cranium
KA1 bone portion
KA2 bone portion
GG external auditory canal
W wall
OS surgical template
FS void
FS1, FS2 void parts
AF contact face
WA wall portion
DF sealing face

1-15. (canceled)

16. A temporal bone implant (SI) for at least partially restoring a posterior wall (W) of an exterior auditory canal (GG), the temporal bone implant (SI) comprising:
a first portion (A1) shaped as a tube or a tube segment, the first portion (A1) configured to form at least one wall portion (WA) of the posterior wall (W) by covering a void (FS) in a temporal bone (SB) and thereby separate the auditory canal (GG) from the void (FS); and
a second portion (A2) that is plate-shaped and oriented transversely to the first portion (A1), the first portion (A1) transitioning into the second portion (A2), the second portion (A2) configured for attachment to a mastoid of the temporal bone (SB) and to cover the void (FS) together with the first portion (A1).

17. The temporal bone implant (SI) of claim 16, wherein:
the second portion (A2) comprises an attachment part (BT) and a cover part (AT),
the attachment part (BT) forms a contact face (AF) on an inner face (IS) of the temporal bone implant (SI);
the temporal bone implant (SI) is placeable on the temporal bone (SB) at the inner face (IS) of the temporal bone implant (SI);
the contact face (AF) is contactable with the mastoid for attachment to the mastoid; and
the cover part (AT) is configured for at least partially covering the void (FS).

18. The temporal bone implant (SI) of claim 17, wherein the attachment part (BT) peripherally partially surrounds the cover part (AT).

19. The temporal bone implant (SI) of claim 17, wherein a plurality of receiving openings (AO) is defined in the attachment part (BT) of the second portion (A2), the plurality of receiving openings (AO) extending from the inner face (IS) to an outer face (AS) of the temporal bone implant (SI) facing away from the inner face (IS), the plurality of receiving openings (AO) configured for receipt of attachment mechanisms.

20. The temporal bone implant (SI) of claim 17, wherein a plurality of passages are defined in the cover part (AT), the plurality of passages extending from the inner face (IS) to an outer face (AS) of the temporal bone implant (SI) facing away from the inner face (IS).

21. The temporal bone implant (SI) of claim 16, wherein:
the first portion (A1) forms a sealing face (DF) on an inner face (IS) of the temporal bone implant (SI);
the temporal bone implant (SI) placeable on the temporal bone (SB) at the inner face (IS) of the temporal bone implant (SI); and
the sealing face (DF) is placeable on the temporal bone (SB).

22. The temporal bone implant (SI) of claim 21, wherein a coating is applied on the sealing face (DF).

23. The temporal bone implant (SI) of claim 21, wherein the sealing face (DF) is at least partially formed on a periphery of the first portion (A1) and is configured for sealing the auditory canal (GG) with respect to the void (FS) via contact with the temporal bone (SB).

24. The temporal bone implant (SI) of claim 16, wherein the first portion (A1) and the second portion (A2) are shaped in a patient-specific manner.

25. The temporal bone implant (SI) of claim 24, wherein the first portion (A1) and the second portion (A2) are modeled after bone portions (KA1, KA2) which are to be removed from the temporal bone (SB) of the patient.

26. A method for configuring and/or manufacturing a temporal bone implant (SI) for at least partially restoring a posterior wall (W) of an exterior auditory canal (GG), the method comprising:
forming a first portion (A1) in the temporal bone implant (SI), the first portion (A1) shaped as a tube or a tube segment and configured to form at least a part of the posterior wall (W) by covering a void (FS) in a temporal bone (SB) and thereby separate the auditory canal (GG) from the void (FS),
forming a second portion (A2) in the temporal bone implant (SI), the second portion (A2) being plate-shaped and oriented transversely to the first portion (A1), the second portion (A2) is attachable to a mastoid of the temporal bone (SB) and configured to cover the void (FS) together with the first portion (A1).

27. The method of claim 26, wherein one or both of the first portion (A1) and the second portion (A2) is modeled after bone portions (KA1, KA2) that are to be removed from the temporal bone (SB) of the patient.

28. The method of claim 26, wherein the sizing and shaping of the first portion (A1) and of the second portion (A2) is determined based on previously determined contours of the temporal bone (SB).

29. The method of claim 26, further comprising configuring and/or manufacturing a surgical template (OS) together with the temporal bone implant (SI).
